(19) United States
(12) Patent Application Publication
(10) Pub. No.: US 2020/0368796 A1
(43) Pub. Date: Nov. 26, 2020

(54) METHOD AND APPARATUS FOR NITROGEN DISINFECTION OF MEDICAL WASTE MATERIAL

(71) Applicants: Julius B. CO, Antelope, CA (US); Roderick S. DAYOT, Davao City (PH)

(72) Inventors: Julius B. CO, Antelope, CA (US); Roderick S. DAYOT, Davao City (PH)

(21) Appl. No.: 16/880,777

(22) Filed: May 21, 2020

Related U.S. Application Data
(60) Provisional application No. 62/850,844, filed on May 21, 2019.

(51) Int. Cl. B09B 3/00; A61L 2/06; A61L 2/26

(52) U.S. Cl. CPC B09B 3/0075; A61L 2/06; A61L 2202/122; B09B 3/0083; A61L 2/26

(57) ABSTRACT

Example embodiments of the present invention relate generally to medical waste management and more specifically to a method and apparatus for treating waste material using hot nitrogen gas as a better alternative non-burn technology.

### PRIORITY PATENT APPLICATION

This non-provisional patent application draws priority from U.S. provisional patent application Ser. No. 62/850,844; filed May 21, 2019. The entire disclosure of the referenced patent application is considered part of the disclosure of the present application and is hereby incorporated by reference herein in its entirety.

### COPYRIGHT

A portion of the disclosure of this patent document contains material that is subject to copyright protection. The copyright owner has no objection to the facsimile reproduction of the patent document or the patent disclosure, as it appears in the U.S. Patent and Trademark Office patent files or records, but otherwise reserves all copyright rights whatsoever. The following notice applies to the disclosure provided herein and to the drawings that form a part of this document: Copyright 2019-2020, Julius B. CO and Roderick S. DAYOT; All Rights Reserved.

### TECHNICAL FIELD

Example embodiments of the present invention relate generally to medical waste management and more specifically to a method and apparatus for treating waste material using hot nitrogen gas as a better alternative non-burn technology.

### BACKGROUND

Waste materials, such as medical waste made of plastic, cloth, paper, cellulose, etc., are usually burned in incinerators where incomplete combustion is the normal process that produces pungent smoke where to counter such result is done with expensive flue gas treatment processes that would ensure environmental safety and protection. Besides the use of incinerators, another non-burning method of treating medical waste is the use of an autoclave wherein high pressure and high temperature steam is produced from the boiler and is used to disinfect the medical waste through direct injection in the waste chamber. The aforesaid introduction of steam to the medical waste has been observed to be insufficient in disinfecting the waste since the distribution of heat and heat transfer is uneven during treatment. Furthermore, the steam being applied converts into water at a fast rate such that the moisture from the steam becomes a wastewater, which requires another treatment, and medical waste with water from steam becomes wet, heavy, and odorous. Another form of treatment of medical waste is the microwave where the waste material is subjected to microwave radiation for heating. The said heating however needs moisture to excite water molecules to produce heat and the temperature produced is 115 degrees Celsius, which is below disinfection temperature that is 121 degrees Celsius requiring more energy to achieve high temperature. In addition, the microwave waste treatment is utilizing a microwave generator that is very sensitive to moisture, can be damaged easily, and is expensive. Yet another form of treatment of medical waste is the pyrolysis method where waste is thermally decomposed in the absence of oxygen during heating within a designated chamber resulting in a byproduct of char or carbonized material. The source of heat is a high temperature plasma torch or burner that directly or indirectly heats the waste. The said treatment however cannot achieve a complete oxygen free environment that compromises the pyrolysis process as oxygen is technically present in the system that creates combustion, which is considered incineration. In matters of incineration, medical and hazardous waste, such as plastic materials and other organic materials have different heating values such that they react differently when subjected to burning, such that exhaust gases discharged from these incinerators contain polluting components, like smoke, dust, hydrogen chloride, carbon monoxide, Sox, Nox, and heavy metals that includes mercury, dioxin and furan, which are considered harmful pollutants. From the standpoint of environmental protection, it is necessary that formation of these harmful substances should be prevented during waste disinfection treatment.

Of these polluting components, dioxin and furan have extremely strong toxicity such that collection and removal of these is extremely important. Plastics from medical waste should therefore have to be conditioned such as drying, shredding and segregating to achieve complete combustion along with the other gases generated by the other organic materials. This will ensure reduction to safety level the toxic gases coming from the exhaust of the incinerator.

Burning of these medical wastes requires high temperature, preferably above 800 to 1200 degrees Celsius, to facilitate complete combustion and elimination of toxic gases generated by such medical waste. However, in conventional incinerators such temperatures cannot be attained since it will require a tremendous amount of fuel, thus rendering it to be too costly to do.

An example of the incinerator of the prior art is the conventional refuse incinerator facility, wherein a boiler and auxiliary burner are used. The refuse is directly burned in order to raise the temperature of the incinerator and the temperature of boiling water in the boiler. At the initial start of the operation, there is already a production of a low-temperature combustion gas which inflicts damage to the facility due to low-temperature corrosion build-up. To solve this problem, the common practice is to discharge this combustion gas by way of a bypass duct and stack. However, there is still the possibility that dust containing hazardous substances, such as dioxin, remain in the incinerator and boiler. If such contamination substances are deposited and still remain in the incinerator, they may be emitted and discharged as gaseous dioxin into the atmosphere even during normal operation.

Another example is an exhaust gas treating apparatus wherein the refuse is incinerated and then completely combusted by a secondary burner in a secondary combustion chamber. The ashes are then discharged to the atmosphere while the exhaust gas generated by the combustion is subjected to heat recovery by a waste heat boiler and waste heat reclaimer (pre-heater) as it flows towards a quenching reaction tower. The exhaust gas in the quenching reaction tower is sprayed with slaked lime slurry to remove hydrogen chloride (HCL) and sulfur oxide (SOx). Smoke dust, fly ash, HCL, SOx, heavy metals and dioxins, which remain in the exhaust gas, are then removed in a bag filter. The exhaust gas after treatment in then discharged to the atmosphere.

Although the exhaust gas from an incinerator is treated with the aforesaid process, there is the possibility that dioxin cannot be reduced to the desired low concentration. Dioxins generated during incineration are almost decomposed in the secondary combustion chamber; however, it is necessary to decrease the temperature of the exhaust gas from a high temperature of about 350 to 900 degree centigrade to a low temperature during every step of the process. However, there is still the tendency of dioxins regenerating at the vicinity of 300 degrees Centigrade during every step of the process such that the above-mentioned conventional exhaust gas treatment apparatus cannot effectively collect and remove dioxins at the desired low concentration.

### SUMMARY

The example embodiments of the present invention as herein disclosed provide treatment of waste materials wherein biodegradable and non-biodegradable waste materials, in particular medical waste, such as plastic, paper, cellulose, etc. are subject to a disinfection process such that high temperature nitrogen gas is utilized as a heat medium to facilitate effective treatment. Nitrogen gas disinfection is defined in the example embodiments of the present invention as the disinfection process of heating infectious medical waste and other waste material elements without oxidation through heat transfer from a hot nitrogen gas environment in which the thermally heated nitrogen gas is introduced into the waste chamber that displaces air and other gas and the waste is effectively disinfected without burning. The novel process reduces the waste into a gas or changes its phase into a gaseous form because the process is without the presence of air and oxygen. Furthermore, the example embodiments of the present invention are capable of disinfecting such waste through utilization of a heater that raises the nitrogen gas temperature generated by the apparatus. The example embodiments of the present invention further utilize an environmentally compatible non-oxidant, non-combustible gas, such as cheap and readily available nitrogen gas in combination with a heating system assisted with an external heater for high temperature disinfection. The gas being emitted by the example embodiments of the present apparatus is pollutant-free since only nitrogen gas is used and no chemicals are added in the process.

In view of the aforesaid problems of the prior art, it is therefore an object of example embodiments of the present invention to provide a method and apparatus for disinfecting waste materials, which can remedy the above-described drawbacks of the prior art including the drawbacks of the present non-burn technologies, such as autoclave, microwave, and pyrolysis methods, which are expensive to manufacture, operate, and maintain.

A further object of the example embodiments of the present invention is to provide an apparatus for treating waste, which can be used for heating and treating other waste materials such as garbage and like materials that require disinfection. The example embodiments of the present invention are very easy to operate and can be used in cities and hospitals for their medical and garbage waste management. Other objects and advantages of the example embodiments of the present invention may be realized upon reading the following description taken in conjunction with the accompanying drawings.

### BRIEF DESCRIPTION OF THE DRAWINGS

Embodiments are illustrated by way of example and not limitation in the figures of the accompanying drawings, in which:

FIG. 1 is an illustrative presentation of an example embodiment of the present invention being used in the disinfection treatment of waste materials;

FIG. 2 is a side view of one of the example embodiments of the present invention;

FIG. 3 is a presentation of another example embodiment of the present invention; and

FIG. 4 is the cross section of the example embodiment of FIG. 3.

### DETAILED DESCRIPTION

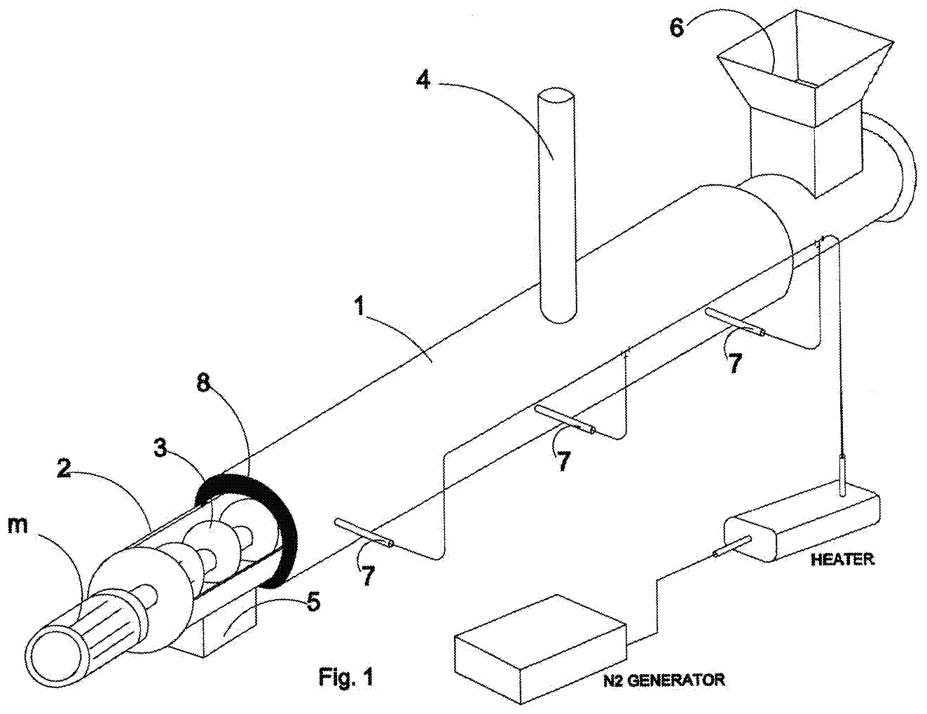

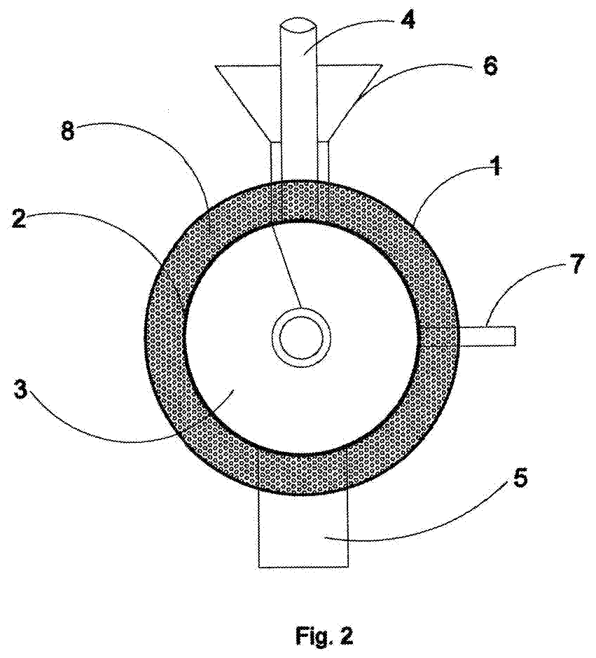

Referring to the drawings, FIGS. 1 and 2, there is shown an apparatus for disinfecting waste, such as medical and hazardous waste and garbage waste, the apparatus generally designated as 1 comprising an insulated structure 8 having a plurality of pipe heating assemblies 7 fixedly held on said insulated structure. Said insulated structure being arranged such that it is capable of being held in an elevated manner by suitable conventional support structure (not shown). Insulated structure 8 includes a stationary shell member 2 and rotary inner shell member 3, and a heat chamber being defined by said outer and inner shell members and disposed there between. Said stationary inner shell member 2 defining a waste heating chamber, wherein the waste material is capable of passing or moving in a rotary motion starting from the input chute and directly heated and at the same time while the waste therein is constantly heated such that it can be transformed into disinfected waste material. A conveying means 3 disposed within inner shell member 2 being held thereof in a manner wherein it is capable of providing movement of waste material being heated for disinfection. Conveying means 3 is preferably a rotary conveyor being capable of rotational movement by suitable prime mover, such as electric motor M. A waste material inlet 6 is provided on one side of the inner shell member 2 being in communication with a waste airlock system fixedly held thereof, and a discharge port 5 provided at the bottom end of said inner shell member 2, wherein the disinfected waste material is being discharged by said conveying means 3. Each of said heating pipe assemblies 7 includes a heating chamber in communication with said heat chamber, a superheated nitrogen producing means N2 being connected to said heat chamber, and fixedly held on said superheated nitrogen producing means H. A heater being arranged in a manner wherein it is capable of providing heat in said superheated nitrogen producing means. Said superheated nitrogen producing means is preferably a heat exchanger being made such that it is capable of transforming nitrogen contained therein into superheated nitrogen through utilization of heat generated by the burner flame or electric heater. The superheated nitrogen producing means being defined by a nitrogen containing means mounted on a nitrogen chamber heater, a superheated nitrogen line 7 having a heat exchange section disposed within the nitrogen heat chamber and in communication with said nitrogen containing means. Nitrogen gas flowing in said superheated nitrogen line 7 being capable of transforming into superheated nitrogen upon exposure to high temperatures generated by the heater through the burner flame or electric heater. Superheated nitrogen line 7 is preferably made of insulated chromium and nickel alloy pipe such that it can serve as medium of the superheated nitrogen into the chamber upon contact thereof. Said gases then being introduced to the treatment chamber through a gas discharge port of the superheated nitrogen line 7. The insulated structure 8 may be made to communicate with at least another identical insulated structure such that further disinfection can be facilitated. An air cooling chamber may be provided and made to communicate with one of said insulated structures to facilitate cooling and disposing the carbonized and/or disinfected waste material coming from the said insulated structure. Furthermore, a gas filtering means may be provided and arranged to communicate with said insulated structure connected by the stack 4 such that filtering of exhaust spent nitrogen gas, produced in the waste chamber during the heating of the waste material, may be discharged as room temperature nitrogen gas. In one arrangement, gas filtering means (not shown) includes a draft fan for drawing the gases, and filtered with an activated carbon filter for filtering the fugitive particulate matter from the nitrogen gas.

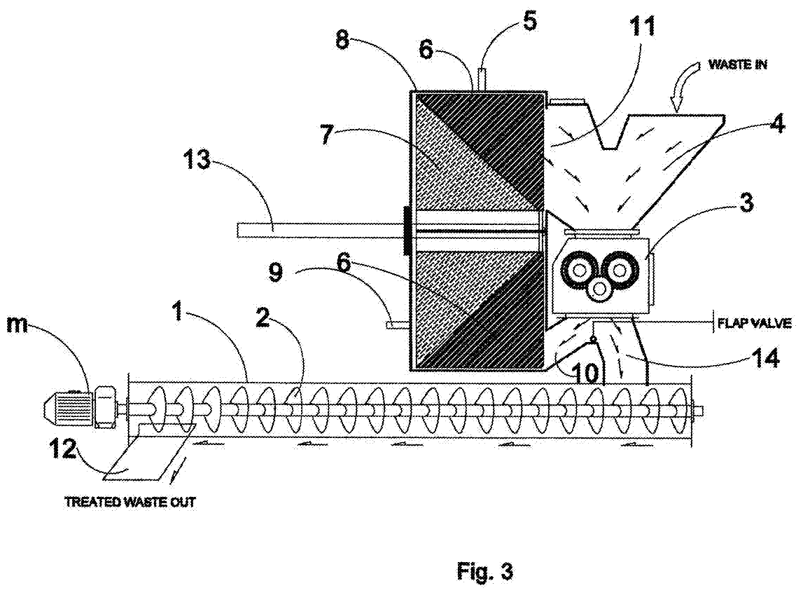

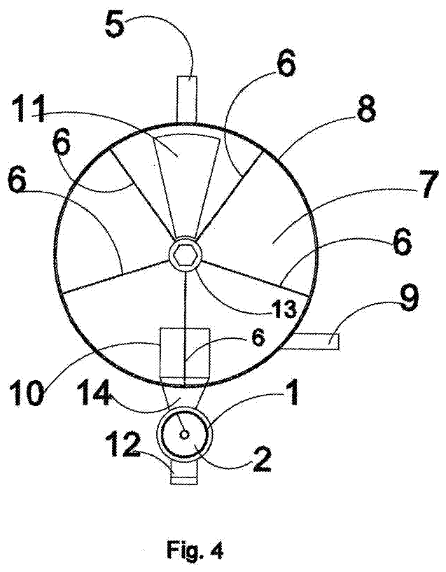

In another embodiment of the present invention described in FIGS. 3 and 4, the apparatus for treating waste material comprises an elevated insulated sealed structure 8 having a parallel coupled and sealed rotary shell mounted therein, which is also a waste heating chamber assembly means in communication with said rotary shell. Waste heating chamber being in communication through a plurality of hot nitrogen gas passages 9 provided on the side section. A hot nitrogen producing means connected to the pipe 9 supplies the hot nitrogen gas. The sealed rotary chamber 7 is comprised of angular paddles which are arranged in a vane like position 6. The vane will transport the waste from the shredder and into the shredder repeatedly until the waste is reduce in size thoroughly through the rotary motion of the rotary shell 7. A hopper 4 is connected to the stationary sealed structure 8 such that it can also be utilized as a waste input medium, which is also connected to the waste shredder under the base of the hopper 4. The waste shredder is a two-shaft multiple teeth system connected to the spout with a diverter: going into the waste heating chamber 10 and going into the waste cooler 14. The rotary shell is driven by a shaft 13 connected to the driving medium such as a motor, a geared motor, or a motor connected to a belt or chain. The cooling chamber 1 is a cylindrical member with a conveying member 2 that transports the waste in communication with the diverter 14 driven by a motor M pushing the waste out of the cooler through an output duct 12.

The method for treating waste material of an example embodiment includes among the following steps or actions. Heat nitrogen at a high temperature until it is transformed into superheated nitrogen. Said heating can be facilitated by suitable burning or heating means, such as a burner or an electric heater. The superheated nitrogen is then allowed to flow within a chromium and nickel pipe connected to the waste treatment chamber means such that heat is generated from said superheated nitrogen. Said high temperature heat inducted/transferred by the hot nitrogen in the heat chamber is then indirectly transferred to the waste heating chamber to facilitate disinfection of the waste being treated. When treating biodegradable and non-biodegradable medical or garbage waste material, such as plastic, wood, paper, cellulose, etc. by means of thermal disinfection, the temperature within the waste heating chamber should be at 120 to 300 degrees Celsius. In case the waste to be treated is infectious medical waste, the heating temperature in the waste heating chamber is regulated at minimum temperature to about 121 degrees Celsius to facilitate disinfection and kill the pathogens in the waste. At this temperature, no pathogens and viruses can survive after disinfection.

We claim:

1. A method for disinfecting waste material, the method comprising:
heating of nitrogen gas at a pre-determined high temperature until it is converted into superheated nitrogen gas, including any non-combustible gas, gases that are stable at high temperature, and inert gas;
allowing said superheated nitrogen gas be in contact with waste material inside a heating chamber;
heating the waste material by said heat of the superheated nitrogen gas; and
disinfecting the waste material utilizing the superheated nitrogen gas with a temperature from 100 to 350 degrees Celsius and a pressure from 10 psi to 50 psi.

2. The method for disinfecting waste material according to claim 1, wherein the temperature of said heat utilize for this apparatus and method is 150 to 300 degree Celsius.

3. The method for disinfecting waste material according to claim 1, wherein said waste material is agricultural biological waste, biohazard matter, biological matter, medical waste, infectious waste, hospital waste, clinical waste and hazardous waste.

4. The method for disinfecting waste material according to claim 3, wherein the temperature of the superheated nitrogen is 121 to 300 degrees Celsius when the waste material being heated is medical waste to disinfect and eliminate viruses and pathogens.

5. The method for disinfecting waste material according to claim 1, wherein the waste material is directly and indirectly heated by said heat from the superheated nitrogen gas.

6. An apparatus for disinfecting waste material, the apparatus comprising:
an insulated structure which is the disinfection chamber having a plurality of heating assemblies being held thereof, each of said heating assemblies being arranged in a manner wherein it is capable of providing heat in said insulated structure, said insulated structure being made such that it is capable of disinfecting and disposing waste materials being heated and disinfected therein, insulated structure includes an insulated hollow cylindrical stationary outer shell member and hollow rotary moving inner shell member that defines a heating chamber disposed there between, a waste heating chamber being defined by said inner rotating shell member on which is also a conveying means provided within the inner shell member being held thereof in a manner wherein it is capable of providing rotary movement and disposal of the heated and/or disinfected waste materials disposed in the inner shell member, each of said heating assemblies consisting of a heat pipe in communication with said heating chamber, a superheated nitrogen producing means in communication with said heating chamber, and a heater heated using a burner or electric source is fixedly held on said superheated nitrogen producing means and being arranged such that it is capable of providing heat in said superheated nitrogen producing means, said superheated nitrogen producing means being capable of transforming cold nitrogen contained therein into superheated nitrogen through utilization of heat generated by said burner or heater.

7. The apparatus for disinfecting waste material according to claim 6, wherein said superheated nitrogen producing means is a heat exchanger or heat transfer medium.

8. The apparatus for disinfecting waste material according to claim 6, wherein said superheated nitrogen line being made of insulated chromium and nickel alloy such that it can serve as best material for effective less resistance flow of hot nitrogen gas.

9. The apparatus for disinfecting waste material according to claim 6 wherein said conveying means is a rotary paddle conveyor, spoke conveyor and screw conveyor.

10. The apparatus for disinfecting waste material according to claim 8, wherein said superheated nitrogen line having insulated section connecting the heating chamber.

11. The apparatus for disinfecting waste material according to claim 6, wherein said insulated structure which is the disinfection chamber being in communication with an identical insulated structure and so on such that further disinfection of waste could be facilitated.

12. The apparatus for disinfecting waste material according to claim 6, wherein a cooling chamber is in communication with one of said insulated structure, which is the disinfection chamber being made such that it is capable of cooling and disposing of the disinfected waste from said insulated structure.

13. The apparatus for disinfecting waste material according to claim 6, wherein said waste material is agricultural biological waste, biohazard matter, biological matter, medical waste, infectious waste, hospital waste, clinical waste and hazardous waste.

14. The apparatus for disinfecting waste material according to claim 6, wherein a gas filtering means is in communication with said insulated structure being made such that it is capable of filtering the gases produced in the said waste chamber during the heating of said waste materials, such as particulate matter, carbon monoxide, carbon dioxide and water.

15. An apparatus for disinfecting waste material, the apparatus comprising:
an insulated structure having a rotary shell being held therein, said rotary shell having a waste heating chamber, in communication with said rotary shell being arranged such that it is capable of providing heat thru the induction of high temperature nitrogen gas to said rotary shell, said heating assembly being defined by a superheated nitrogen producing means having a burner and/or electric resistance coil or any radiation source and light source.

16. The apparatus for treating waste material according to claim 15, wherein said superheated nitrogen producing means is a heat exchanger heated by burner or electrical energy, solar and radiation heat source.

17. The apparatus for treating waste material according to claim 15, wherein said superheated nitrogen is being introduced into the chamber thru single or multiple ports with flow control system.

18. The apparatus for disinfecting waste material according to claim 15, wherein said rotary shell is capable of rotation by a prime moving means.

19. The apparatus for disinfecting waste material according to claim 15, wherein said prime moving means is a sprocket chain assembly, geared motor or belt drive.

20. The apparatus for disinfecting waste material according to claim 15, wherein said superheated nitrogen line is connected to the heating chamber.
